(19) United States
(12) Patent Application Publication
(10) Pub. No.: US 2002/0062043 A1
(43) Pub. Date: May 23, 2002

(54) Process for preparing optionally substituted biphenylcarbonyl chlorides

(76) Inventors: Lars Rodefeld, Leverkusen (DE); Alexander Klausener, Pulheim (DE); Friedrich-Wilhelm Ullrich, Bergisch Gladbach (DE)

Correspondence Address: BAYER CORPORATION, PATENT DEPARTMENT, 100 BAYER ROAD, PITTSBURGH, PA, 15205, US

(21) Appl. No.: 10/052,985

(22) Filed: Nov. 7, 2001

(30) Foreign Application Priority Data
Nov. 9, 2000 (DE) 10055498.9

(51) Int. Cl. C07C 51/58

(52) U.S. Cl. 562/859

(57) ABSTRACT

Optionally substituted biphenylcarbonyl chlorides are obtained in an economically and ecologically advantageous manner by reacting biphenyls with oxalyl chloride in a molar ratio of biphenyl to oxalyl chloride of from 0.7 to 1.5 in the presence of a Lewis acid.

### BACKGROUND OF THE INVENTION

The present invention relates to a process for preparing optionally substituted biphenylcarbonyl chlorides from the corresponding biphenyls and oxalyl chloride.

Optionally substituted biphenylcarbonyl chlorides are important intermediates in the preparation of crop protection agents and pharmaceuticals (see, for example, EP-A 683,156).

Processes for preparing optionally substituted biphenylcarbonyl chlorides are already known. Thus, optionally substituted biphenyl can be acylated, giving a biphenyl methyl ketone that is oxidized to the corresponding carboxylic acid, finally giving, by chlorination, the desired biphenylcarbonyl chloride (see Gazz. Chim. Ital. 79, 453 (1949)).

It is also possible to react biphenyls with chloroformamide to give biphenylcarboxamides that are hydrolyzed to the corresponding carboxylic acid, finally giving, again after chlorination, biphenylcarbonyl chlorides (see Angew. Chemie 61, 163 (1949)).

According to a third route, a cyanobiphenyl compound is initially prepared which is hydrolyzed to the corresponding acid and the latter is converted by chlorination into the desired acid chloride (Synthesis 1991, 441 and CA 1958, 7233).

All these processes have the disadvantage of involving three steps, with the associated use of a large number of reactants and auxiliaries. Not only the provision of reactants and auxiliaries but also the disposal of their subsequent products involve great expense. Therefore, all known processes for preparing biphenylcarbonyl chlorides are problematic from an economical and ecological point of view.

Accordingly, a simple, cost-effective and ecologically advantageous process for preparing biphenylcarbonyl chlorides is still needed.

Reactions of aromatic compounds and diphenyl compounds with oxalyl chloride, too, have been described. However, very different reaction products have been obtained, for example, diarylethanediones (see J. Org. Chem. 59, 635 (1994)), diaryl ketones (see Tetrahedron Lett. 36 5209 (1999)), biscarbonyl chlorides (see Chem. Ber. 122, 2291 (1989)) and, starting from benzene and oxalyl chloride, benzoyl chloride or benzophenone, depending on how the reaction is carried out (Ber. 41, 3566 (1908)).

It has also been observed that, on aqueous work-up, it is frequently not the carbonyl chloride that is obtained but the product of its hydrolysis (the corresponding carboxylic acid) (see Org. Synth. Coll. Vol. V, 706 (1973)) and Friedel-Crafts and Related Reactions (III) p. 1259 (1964)).

Thus, the situation is very complicated, and it is impossible to predict which reaction products can be expected for the reaction of optionally substituted biphenyls with oxalyl chloride.

### SUMMARY OF THE INVENTION

This invention, accordingly, provides a process for preparing optionally substituted biphenylcarbonyl chlorides of the formula (I)

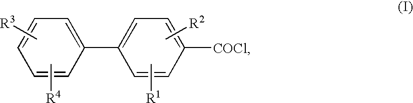

in which

R¹, R², R³, and R⁴ independently of one another each represent

hydrogen, halogen, C₁-C₅-alkyl, or C₁-C₅-alkoxy, comprising reacting a biphenyl of the formula

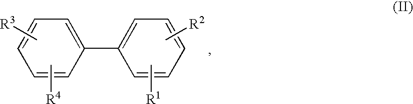

in which R¹, R², R³, and R⁴ are each as defined as for formula (I), with oxalyl chloride in a molar ratio of biphenyl of the formula (II) to oxalyl chloride of from 0.7 to 1.5 in the presence of a Lewis acid.

### DETAILED DESCRIPTION OF THE INVENTION

In the formulas (I) and (II), the radicals R¹ to R⁴ independently of one another each preferably represent hydrogen, fluorine, chlorine, bromine, methyl, ethyl, methoxy, or ethoxy.

Furthermore, R¹ and R² each preferably represent hydrogen and R³ and R⁴ each preferably represent hydrogen, fluorine, chlorine, bromine, methyl, ethyl, methoxy, or ethoxy.

It is furthermore preferred if R¹, R², and R³ each represent hydrogen and R⁴ represents fluorine, chlorine, methyl, ethyl, methoxy, or ethoxy in the p-position.

Particularly preferably, R¹, R², and R³ each represent hydrogen and R⁴ represents fluorine or chlorine in the p-position.

Biphenyis of the formula (II) can be obtained by a large number of different ways or analogously to known processes and are also commercially available (see, e.g., Houben-Weyl, Vol. V/2b, 224 (1981), Org. Synth. Coll. Vol. V, 51 (1978), Chem. Ber. 95, 2469 (1995), and Synth. Comm. 29, 4423 (1999)).

The molar ratio of biphenyl of the formula (II) to oxalyl chloride is preferably from 0.9 to 1.1. Particular preference is given to using equi-molar amounts.

Lewis acids suitable for the process according to the invention are, e.g., the chlorides of boron, aluminum, phosphorous, antimony, iron, zinc, and tin. Preference is given to aluminum chloride.

Based on the oxalyl chloride the Lewis acid can be employed, e.g., in a molar ratio of from 0.9 to 2.5. This ratio is preferably from 1 to 1.5, in particular from 1 to 1.2.

The process according to the invention can be carried out in the presence of a solvent. Suitable solvents are, e.g., polychlorinated alkanes, such as methylene chloride, dichloroethane, and tetrachloroethane, and aromatic compounds for which the reactivity with oxalyl chloride is lower than that of the biphenyl to be reacted, such as, for example, chlorobenzene or o-dichlorobenzene. It is, of course, important to use only those solvents that are liquid under the reaction conditions in question.

The process according to the invention can be carried out, for example, at temperatures in the range from −30 to +80° C. Preference is given to temperatures in the range of from −20 to +60° C., in particular to those in the range from −10 to +40° C.

The process according to the invention can be carried out as desired. It is possible, for example, to initially charge the Lewis acid, the biphenyl of the formula (II), and the solvent, and to carry out a metered addition of the oxalyl chloride, if appropriate together with further solvent, to the initially charged mixture. It is also possible to initially charge a mixture of Lewis acid and solvent, followed by a metered addition of a mixture of biphenyl of the formula (II) and oxalyl chloride, and if appropriate together with further solvent, to the initial charge. Simultaneous metered addition of the oxalyl chloride and a solution of the biphenyl of the formula (II) in the solvent to the Lewis acid, initially charged with solvent, is likewise possible. Further ways of carrying out the process according to the invention are also conceivable.

Once the addition of reactants, catalyst and, if appropriate, solvent is complete, it is generally advantageous to continue stirring at a temperature in the range from −10 to +40° C. for a while.

Work-up of the mixture that is present after the reaction and, if appropriate, after the extra stirring time has lapsed, can be carried out, for example, by metering the entire reaction mixture into a mixture of ice and a mineral acid, removing the aqueous phase, and isolating the resulting biphenylcarbonyl chloride from the organic phase. Depending on the demands on purity that the resulting biphenylcarbonyl chloride must meet, a crystallization for further purification of the crude product may also follow, if required. Solvents suitable for such a crystallization are slightly polar to nonpolar hydrocarbons. Examples of suitable solvents are alkanes such as hexane, heptane, octane, nonane, or decane, including all possible isomers, and also mixtures of these compounds.

Work-up of the reaction mixture is advantageously carried out in strongly acidic medium. Under such conditions, the salts formed from the Lewis acid remain in solution. The preferred mineral acid is hydrochloric acid. If a freshly prepared mixture of ice and hydrochloric acid is used, the temperature of this mixture may also be below 0° C. In this case, the temperature increases during work-up. The temperature of the mixture that is worked up is advantageously kept in the range from −20 to +80° C., in particular in the range from −5 to +50° C., as long as water is still present.

The process according to the invention has the advantage that it provides, in only one reaction step and thus in an economically and ecologically advantageous manner, biphenylcarbonyl chlorides in good yields and purities.

The following examples further illustrate details for the process of this invention. The invention, which is set forth in the foregoing disclosure, is not to be limited either in spirit or scope by these examples. Those skilled in the art will readily understand that known variations of the conditions of the following procedures can be used. Unless otherwise noted, all temperatures are degrees Celsius and all percentages are percentages by weight.

### EXAMPLES

### Example 1

50 ml of o-dichlorobenzene, 95.3 g of p-chlorobiphenyl, and 70.7 g of aluminum chloride were initially charged in a round-bottom flask fitted with stirrer, dropping funnel, and gas outlet connected to a downstream scrubber filled with activated carbon and operated with water, and the mixture was cooled to 10° C. Over a period of 30 minutes, 70.2 g of oxalyl chloride were added dropwise, the temperature being kept at 10° C. After the addition had ended, the mixture was stirred at 10° C. for 1 hour and then at 40° C. for 2 hours. For work-up, the mixture was introduced with stirring into a mixture of 250 g of ice and 164 g of 37% by weight strength aqueous hydrochloric acid. Some precipitated solid was removed by filtration. The resulting mother liquor separated into two phases. From the organic phase, after drying with sodium sulfate, concentration and crystallization of the residue from petroleum ether, 77% of theory of 4-chlorobiphenyl-4′-carbonyl chloride were isolated.

### Example 2

50 ml of o-dichlorobenzene, 95.3 g of p-chlorobiphenyl, and 64.7 g of oxalyl chloride were initially charged in a round-bottom flask as used in Example 1, and the mixture was cooled to 0° C. Over a period of 15 minutes, a stirred suspension of 70.7 g of aluminum chloride in 200 ml of o-dichlorobenzene was added dropwise, the temperature being kept at 0° C. After the addition had ended, the mixture was stirred at 0° C. for 1 hour and then at 20° C. for another hour. With stirring, the mixture was introduced into a mixture of 500 g of ice and 164 g of 37% by weight strength aqueous hydrochloric acid. Analytical examination of the organic phase that formed showed that it contained 92% of theory of 4-chlorobiphenyl-4′-carbonyl chloride.

### Example 3

200 ml of o-dichlorobenzene and 70.7 g of aluminum chloride were initially charged in a round-bottom flask as used in Example 1, and the mixture was cooled to 0° C. At this temperature, a solution of 95.3 g of p-chlorobiphenyl and 64.7 g of oxalyl chloride in 350 ml of o-dichlorobenzene was added dropwise over a period of 20 minutes. The mixture was then stirred at 0° C. for 1 hour and at room temperature for another hour. With stirring, the reaction mixture was then poured into a mixture of 500 g of ice and 164 g of 37% by weight strength aqueous hydrochloric acid. The resulting aqueous phase was separated off and washed once more with 250 ml of o-dichlorobenzene. Organic phase and the washed liquid were combined and the resulting mixture was subjected to vacuum distillation. Initially, a water-dichlorobenzene mixture was distilled off. The distillation was then interrupted to filter off small amounts of solid products that had formed. The distillation was then continued up to a bottom temperature of 120° C. and a pressure of 19 mbar. The product melt that remained in the bottom was cooled to 100° C., and 250 ml of petroleum ether were added. On cooling to 4° C., the desired product crystallized. Filtration with suction and drying gave 118.9 g of 4-chlorobiphenyl-4′-carbonyl chloride of a purity of 98.6%. This corresponds to a yield of 93% of theory. The mother liquor contained a further 4% of theory of 4-chlorobiphenyl-4′-carbonyl chloride.

### Example 4

200 ml of o-dichlorobenzene and 70.7 g aluminum chloride were initially charged in a round-bottom flask as used in Example 1, and the mixture was cooled to −5° C. At this temperature, 64.7 g of oxalyl chloride were metered in over a period of 15 minutes. Over a period of 1 hour, a solution of 95.3 g of p-chlorodiphenyl in 350 ml of o-dichlorobenzene was added dropwise to this suspension, at a temperature of at most +5° C. The mixture was then stirred at +5° C. for 1 hour and at room temperature for another hour. With vigorous stirring, the reaction mixture was then poured into a mixture of 500 g of ice and 164 g of 37% by weight strength aqueous hydrochloric acid. The aqueous phase was separated off and washed with 250 ml of o-dichlorobenzene. Organic phase and wash liquid were combined, and this mixture was concentrated by vacuum distillation. Crystallization and work-up according to Example 3 gave 4-chlorodiphenyl-4′-carbonyl chloride in a yield of 93% of theory.

What is claimed is:

1. A process for preparing substituted or unsubstituted biphenylcarbonyl chlorides of the formula (I) 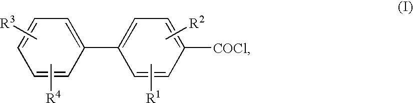
in which
R¹, R², R³, and R⁴ independently of one
another each represent
hydrogen, halogen, C₁-C₅-alkyl, or C₁-C₅-alkoxy, comprising reacting a biphenyl of the formula 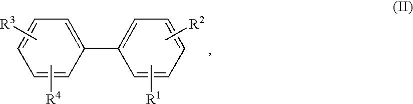
in which R¹, R², R³, and R⁴ are each as defined as for formula (I), with oxalyl chloride in a molar ratio of biphenyl of the formula (II) to oxalyl chloride of from 0.7 to 1.5 in the presence of a Lewis acid.

2. A process according to claim 1 wherein the radicals R¹ to R⁴ independently of one another represent hydrogen, fluorine, chlorine, bromine, methyl, ethyl, methoxy, or ethoxy.

3. A process according to claim 1 wherein R¹, R², and R³ each represent hydrogen and R⁴ represents fluorine, chlorine methyl, ethyl, methoxy, or ethoxy in the p-position.

4. A process according to claim 1 wherein the molar ratio of biphenyl of the formula (II) to oxalyl chloride is from 0.9 to 1.1.

5. A process according to claim 1 wherein the Lewis acid is a chloride of boron, aluminum, phosphorous, antimony, iron, zinc, or tin.

6. A process according to claim 1 wherein from 0.9 to 2.5 mol of Lewis acid are employed per mole of oxalyl chloride.

7. A process according to claim 1 carried out in the presence of a solvent.

8. A process according to claim 1 carried out at temperatures in the range from −30 to +80° C.

9. A process according to claim 1 wherein a mixture of the biphenyl of the formula (II) and oxalyl chloride is metered into an initial charge of the Lewis acid and solvent.

10. A process according to claim 1 wherein the reaction mixture is worked up by metered addition to a mixture of ice and a mineral acid, removal of the aqueous phase, and isolation of the resulting biphenylcarbonyl chloride from the organic phase.
